(19) United States
(12) Patent Application Publication
(10) Pub. No.: US 2016/0153022 A1
(43) Pub. Date: Jun. 2, 2016

(54) INDIVIDUALLY TRACEABLE MULTI-FUNCTIONAL CARRIER PARTICLES FOR VALIDATION OF CONTINUOUS FLOW THERMAL PROCESSING OF PARTICLE-CONTAINING FOODS AND BIOMATERIALS

(71) Applicant: Aseptia, Inc., Raleigh, NC (US)

(72) Inventors: Josip Simunovic, Raleigh, NC (US); Pablo Marcelo Coronel, Apex, NC (US)

(21) Appl. No.: 14/957,271

(22) Filed: Dec. 2, 2015

Related U.S. Application Data
(60) Provisional application No. 62/086,684, filed on Dec. 2, 2014.

(51) Int. Cl. C12Q 1/22; A23L 3/18; G01N 33/02

(52) U.S. Cl. CPC C12Q 1/22; G01N 33/02; A23L 3/18; A23V 2002/00

(57) ABSTRACT

This disclosure is directed to carrier particles. In one possible configuration and by non-limiting example, the carrier particles are individually traceable multi-functional carrier particles for validation of continuous flow thermal processing of particle-containing foods and biomaterials.

### CROSS-REFERENCE TO RELATED APPLICATION

This application claims priority to U.S. application Ser. No. 62/086,684 titled INDIVIDUALLY TRACEABLE MULTI-FUNCTIONAL CARRIER PARTICLES FOR VALIDATION OF CONTINUOUS FLOW THERMAL PROCESSING OF PARTICLE-CONTAINING FOODS AND BIOMATERIALS, filed on Dec. 2, 2014, the disclosure of which is hereby incorporated by reference in its entirety.

### SUMMARY

In general terms, this disclosure is directed to carrier particles. In one possible configuration and by non-limiting example, the carrier particles are individually traceable multi-functional carrier particles for validation of continuous flow thermal processing of particle-containing foods and biomaterials. Various aspects are described in this disclosure, which include, but are not limited to, the following aspects.

One aspect is a fabricated device designed to simulate food particle containing food or biomaterial products being processed using continuous flow thermal sterilization, comprising: polymer carrier structure with conservative flow (faster than any real food/biomaterial particle contained in the product) and thermal (slower heating than any real food/biomaterial particle contained in the product); an identifier for marking and/or coding individual particle shells using letters, numbers, symbols and/or color codes; at least one primary implant enabling the tracking and residence time measurement while travelling through the processing system in real time; at least one secondary implant containing viable microbial cells or spores, enzymes, DNA, RNA or other sub-cellular entities; and at least one indicator enabling determination of viability or inactivation of at least one of the secondary implants following incubation or chemical treatment.

Another aspect is a method of determination of sterility and/or proper processing of particle containing foods, the method comprising: preparing at least one simulated particle; inserting the at least one simulated particle into a continuous flow thermal processing system capable of sterilization of particle containing food or biomaterials; monitoring movement of the at least one simulated particle through the processing system using at least one monitoring detection station/sensor or sensor array; capturing the at least one simulated particle following insertion into the processing system and exposure to a representative thermal processing treatment; incubating the at least one captured simulated particle for a sufficient time and at a sufficient temperature to cause growth or chemical state change of at least one biological entity; and determining sterility status of the processed product by evaluating the growth or absence of growth or chemical change in a secondary implant.

Another aspect is a sterilized shelf stable food or biomaterial product obtained by implementing one or more of the processes or methods described herein.

### BRIEF DESCRIPTION OF THE DRAWINGS

FIG. 1 is a perspective view of examples of several differently sized simulated particles as well as examples of food particles. In this example the food particles are kernels of corn.

FIG. 2 illustrates exemplary aspects of the present disclosure.

FIG. 3 illustrates another exemplary aspect of the present disclosure.

FIG. 4 illustrates another exemplary aspect of the present disclosure.

FIG. 5 illustrates an exemplary system including example simulated particles as well as food particles in the form of green bean particles.

FIG. 6 illustrates the exemplary system including the example simulated particles and food particles shown in FIG. 5, immersed in a liquid.

FIG. 7 is another view of the example simulated particle and food particles.

FIG. 8 is a graph showing the normalized temperatures using the exemplary system shown in FIGS. 5 and 6.

FIG. 9 illustrates examples of various simulated particles.

FIG. 10 is a chart depicting the achieved residence times for the particles shown in FIG. 9.

FIG. 11 is a graph illustrating the achieved residence times and selection of the optimal combination of polymers to achieve the most conservative flow characteristics.

FIG. 12 illustrates an example residence time distribution confirmation.

FIG. 13 illustrates an example set of simulated particles being collected from a food product.

FIG. 14 illustrates a rack for holding the simulated particles.

FIG. 15 illustrates an example bottom particle assembly component and an example of a real-time detectable implant.

FIG. 16 illustrates an example processing system including a feed tank, a hold tube, and a cooling section.

FIG. 17 illustrates an example plant, and plant instrumentation.

FIG. 18 illustrates an example of time-temperature monitoring and reconstruction for each particle.

FIG. 19 further illustrates an example of time-temperature monitoring and reconstruction for each particle.

FIG. 20 illustrates an example temperature history.

FIG. 21 illustrates reconstruction of temperature histories for each test particle that were carried out to observe each individual segment (F₀) accumulation for fluid, bulk and worst-case.

FIG. 22 illustrates an example of a top particle assembly component and a post-process detectable implant.

FIG. 23 illustrates another example of a portion of a simulated particle.

FIG. 24 illustrates examples of simulated particles. More particularly, FIG. 24 shows a particle having a first color (“no growth”) and a particle having a second color (“growth”).

FIG. 25 illustrates other examples of simulated particles.

FIG. 26 illustrates other examples of simulated particles.

FIG. 27 illustrates other examples of simulated particles.

FIG. 28 illustrates other examples of simulated particles.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

There are several approaches to validation of multiphase aseptic processes (i.e. aseptic or continuous flow thermal sterilization of particle containing food or biomaterial products).

A main problem with the validation of these processes is that while it is relatively simple to validate a batch-sterilized (retorted or hot filled) product for safety using stationary placed temperature monitoring/recording probes such as thermocouples or resistance based thermometers, these probes are wired and inappropriate for use under continuous flow conditions. The objective of the safety validation is to prove that the slowest heating, fastest moving element of the product has been appropriately thermally treated, i.e. that it has received a sufficient cumulative level of thermal treatment to achieve inactivation of minimally 10̂12 spores of proteolytic strains of Clostridium botulinum, the most heat resistant and the most toxic microorganism of all human pathogens. Shelf stable (ambient temperature stable) low acid food products are typically processed to an even higher level in order to also inactivate the more resistant spores of spoilage causing (but non-pathogenic) microorganisms such as Clostridium sporogenes, Geobacillus stearothermophillus and Bacillus subtilis—and these microorganisms organisms, being more thermo-resistant as well as non-hazardous for human health are typically used as surrogates for validation of thermal sterilization processing.

At least some embodiments according to the present disclosure include one or more devices that are functional simulated carrier particles which allow for the concurrent identification before and after a test run (using one or more of: visible color, numeric, character, and symbolic markings as well as optionally remotely detectable identity tags such as RFID), real time flow monitoring using the remotely detectable magnetic tag implants as well as post-process bio-load based cumulative lethality validation for each individually traceable particle through the use of hermetically sealed spore suspension of Geobacillus stearothermophillus, preferably also incorporating a color-changing indicator to indicate growth upon incubation (i.e. survival of the process by at least one viable spore) or non-growth (i.e. complete inactivation of the spore population present in the suspension).

The present disclosure includes a system of optimized (conservative flow and heat penetration properties) implant-carrying simulated food particles for monitoring and validation of product and process safety for aseptically processed products containing large solid pieces such as chunky soups, stews and salsas and free and hermetically sealed primary and secondary implants for the first time enabling concurrent real-time flow monitoring, time-temperature exposure history for each individually marked and identifiable test particle with the related post process determination of received cumulative thermal lethality through the use of small precise volumes of hermetically sealed spore suspensions with a predetermined load of bacterial spore surrogates (Geobacillus stearothermophillus, Clostridium sporogenes, Bacillus subtilis) and color-changing indicator for post-process bio-validation required by the regulatory agencies for validation of low acid shelf stable foods.

Some embodiments include one or more of the following, including combinations thereof:

optimized/conservative flow and heat penetration characteristics of implant carrier particles;

individual test particle tagging or marking (pre-process) and identification (pre-process and post-process);

primary implants for real time monitoring of time-temperature history of exposure;

secondary post-process confirmation implant/bio-validation of sterility by incubation of bacterial spores; and

color indicator to determine inactivation or growth of bio-indicator organisms.

At least some embodiments include each of the foregoing.

At least some embodiments provide superior numeric and biological characterization and the resulting better understanding of the process, enabling its optimization and a higher degree of process and product safety at a significantly lower total cost and within a reduced period of time.

A) Establishment of conservative characteristics for fabricated polymer carrier particles:

Design and experimental confirmation of thermally conservative properties—slower heating in heaters and hold tubes.

A) 1. Thermally Conservative Design

Particle shells do not heat in microwave (MW-transparent polymer plastics).

Particles are made of a material that is less thermally conductive than the lowest conductive food particle.

Particle wall thickness designed to provide conservative insulation properties compared to the real food particles.

Particles are equal or larger than the largest expected food particle.

A) 2. Thermally Conservative—Experimental Confirmation

Under conventional heating: measurement of concurrent heat penetration into fabricated particles vs. real food particles was carried out.

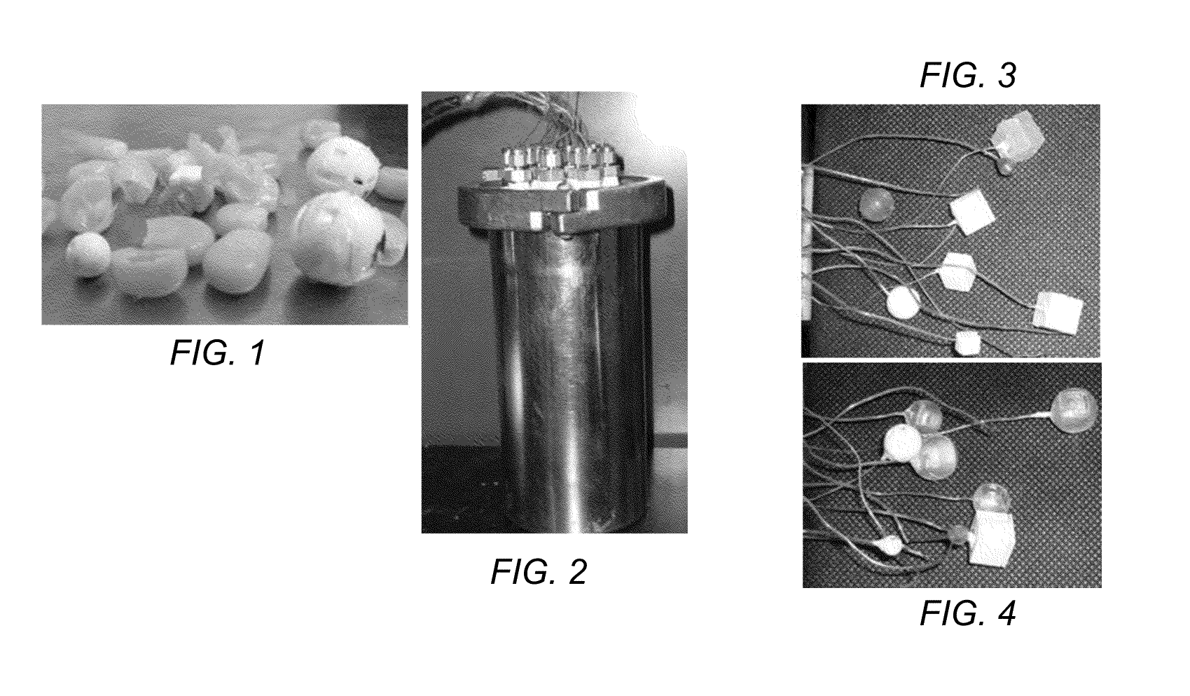

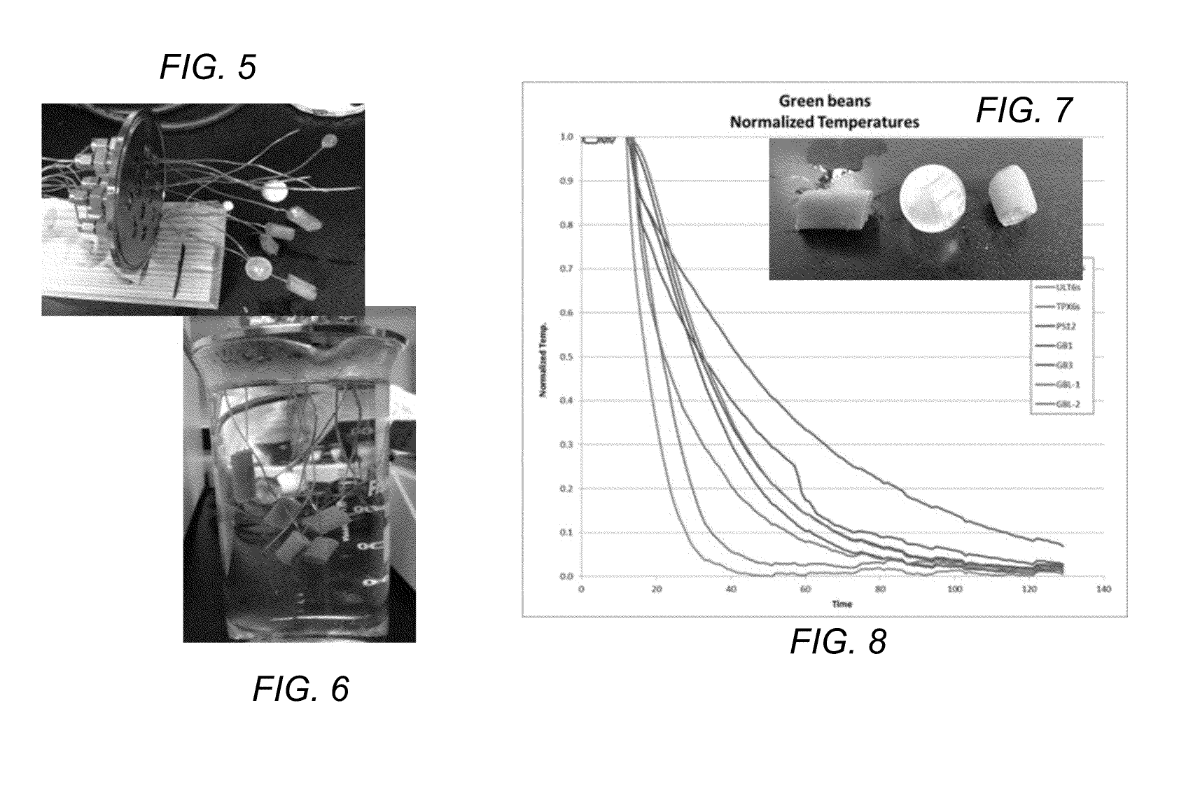

Various aspects are illustrated in FIGS. 1-8.

A) 3. Establishment of Conservative Characteristics for Fabricated Polymer Carrier Particles.

Design and experimental confirmation of flow conservative (fastest moving) properties.

FLOW CONSERVATIVE Design:

Particles were built in 16 combinations of 4 polymers

Effective densities ranged from 0.75 to 1.11 g/cm3

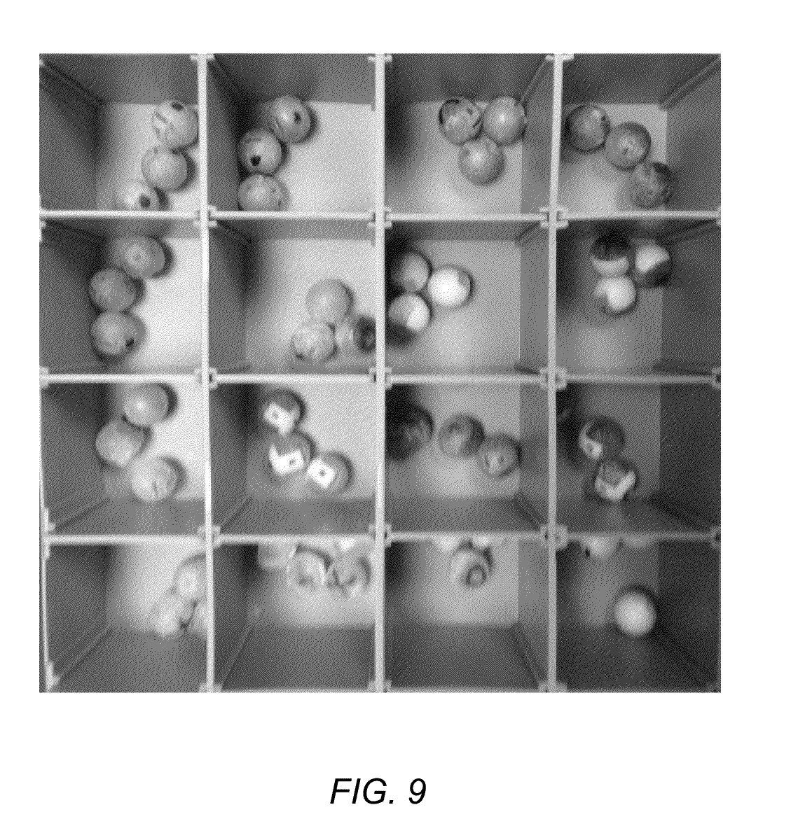

An example is illustrated in FIG. 9.

A) 4. Flow Conservative—Experimental Confirmation

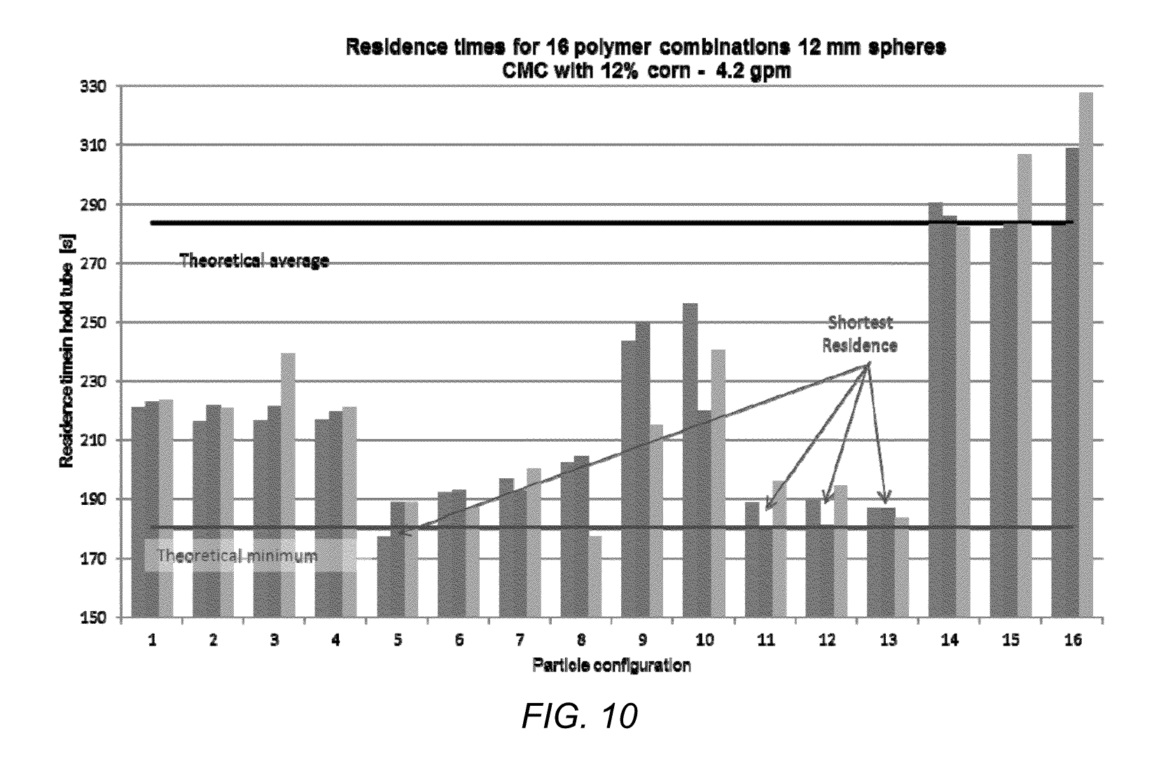

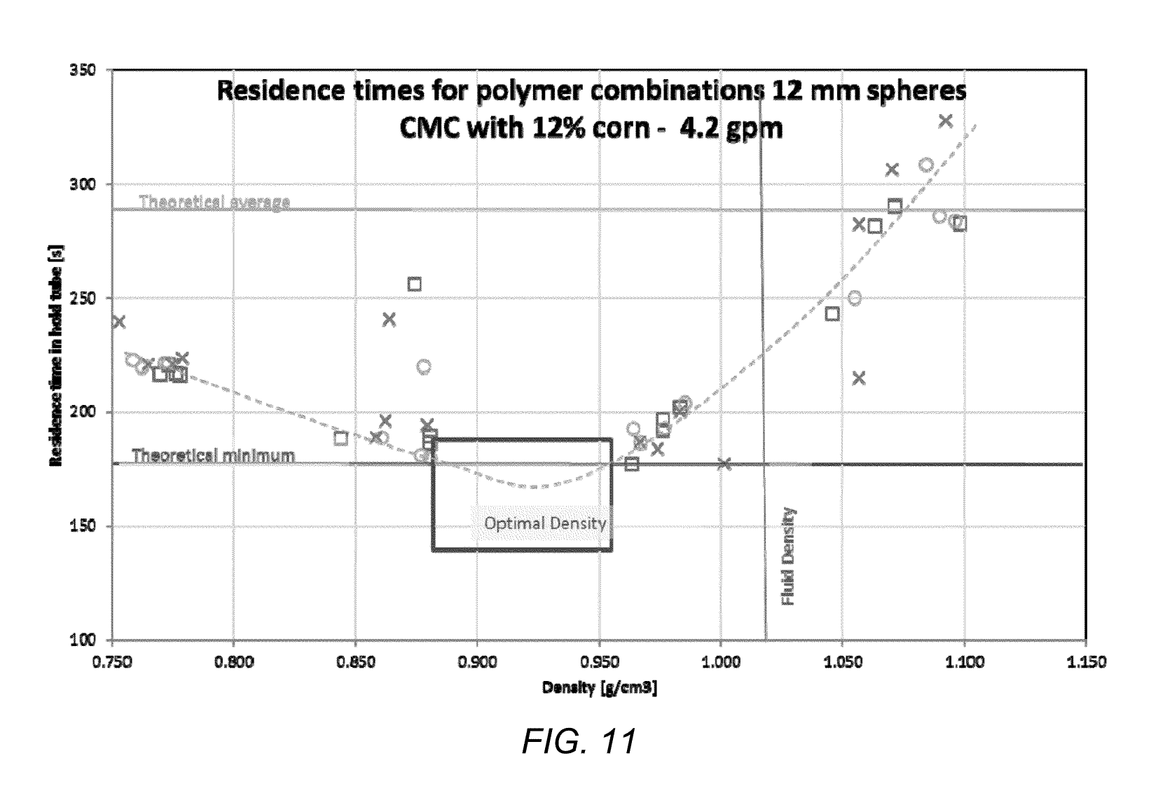

All 16 configurations have been run through the processing system using the representative product environment, as well as representative flow rate, temperature processing profiles and back pressures. FIGS. 10 and 11 illustrate the achieved residence times and the selection of the optimal combination of polymers to achieve the most conservative (fastest moving) flow characteristics.

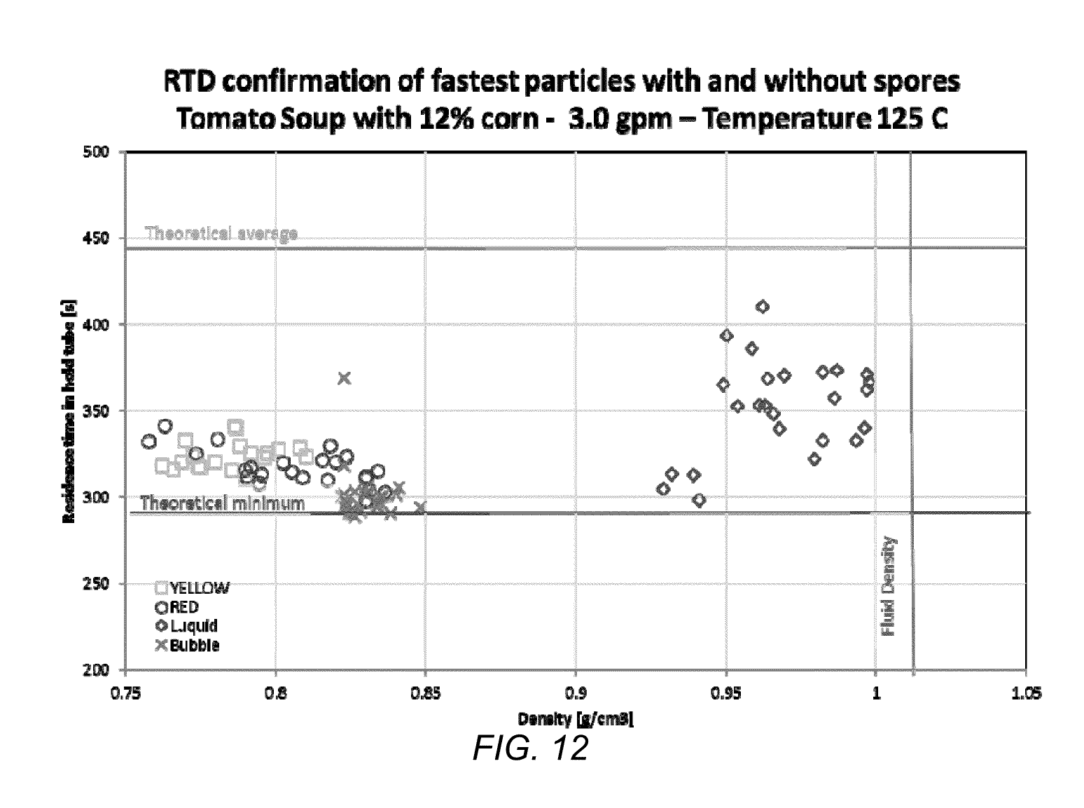

FIG. 12 illustrates an example residence time distribution confirmation of fastest particles with and without spores in tomato soup with 12% corn, 3.0 gpm, at a temperature of 125 degrees C.

B) Individual test particle tagging or marking

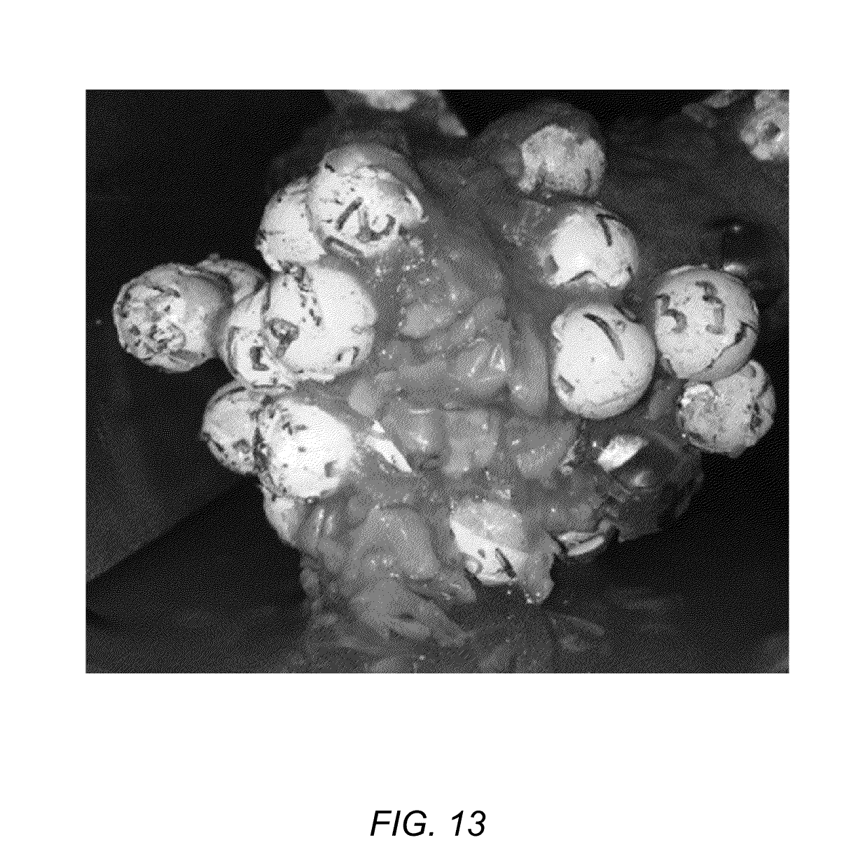

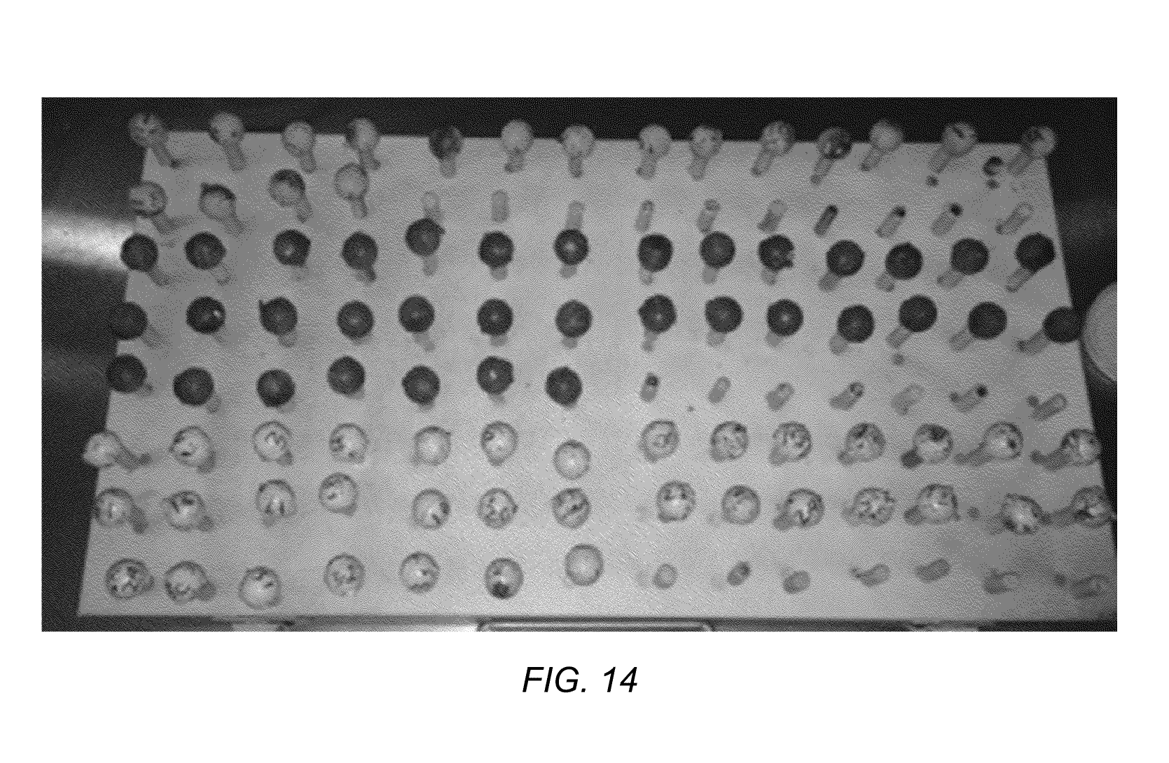

Each particle used is marked with a unique identifiable combination of letters, numbers, symbols and/or color codes. The identification codes are used to keep track of individual characteristics of each particle, times of insertion into the processing system, individual time-temperature histories recorded and post process incubation results. Several example aspects are illustrated in FIGS. 13-14.

C) Primary (magnetic) implants for real time monitoring and reconstruction of time-temperature history of exposure for each individual particle.

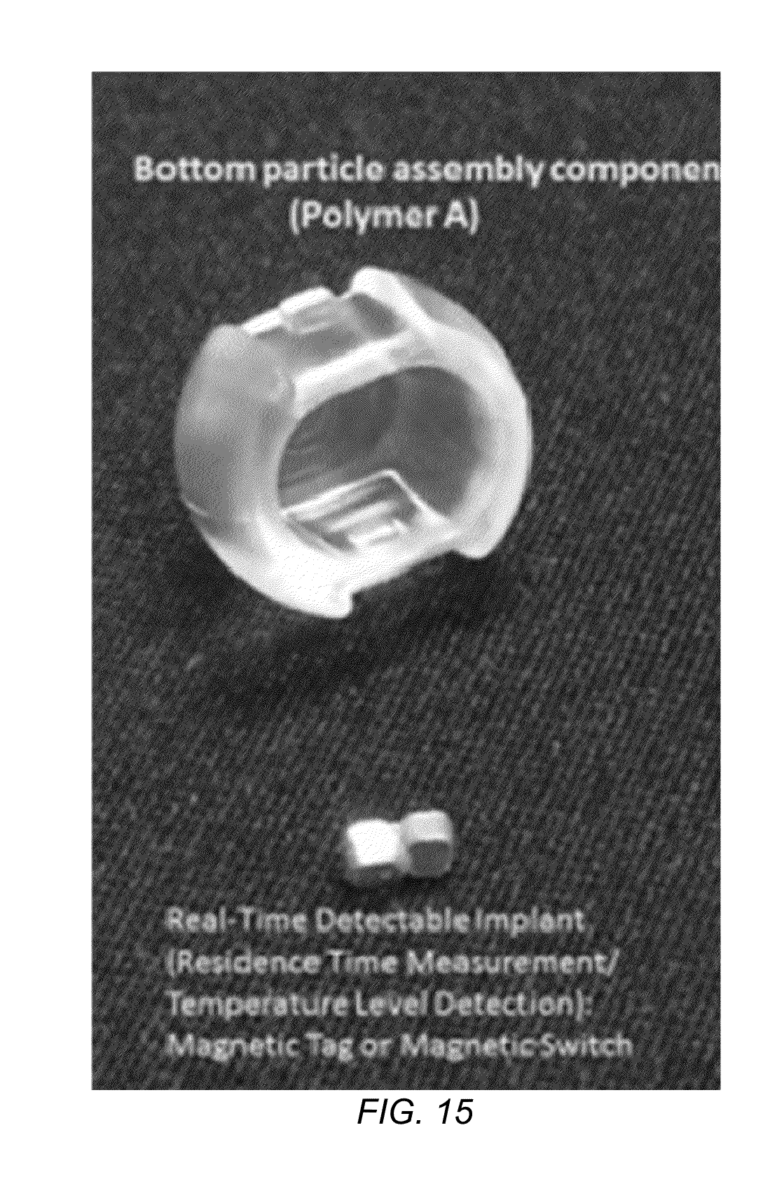

FIG. 15 illustrates an example bottom particle assembly component and an example of a real-time detectable implant.

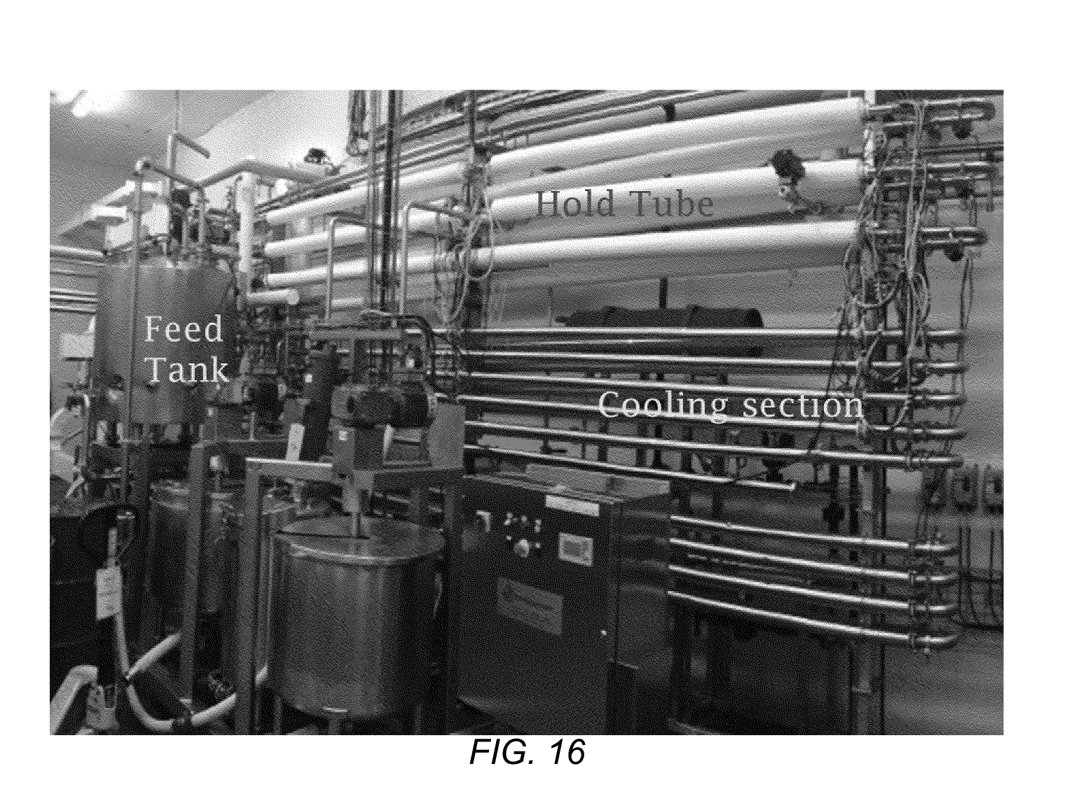

FIG. 16 illustrates an example processing system including a feed tank, a hold tube, and a cooling section.

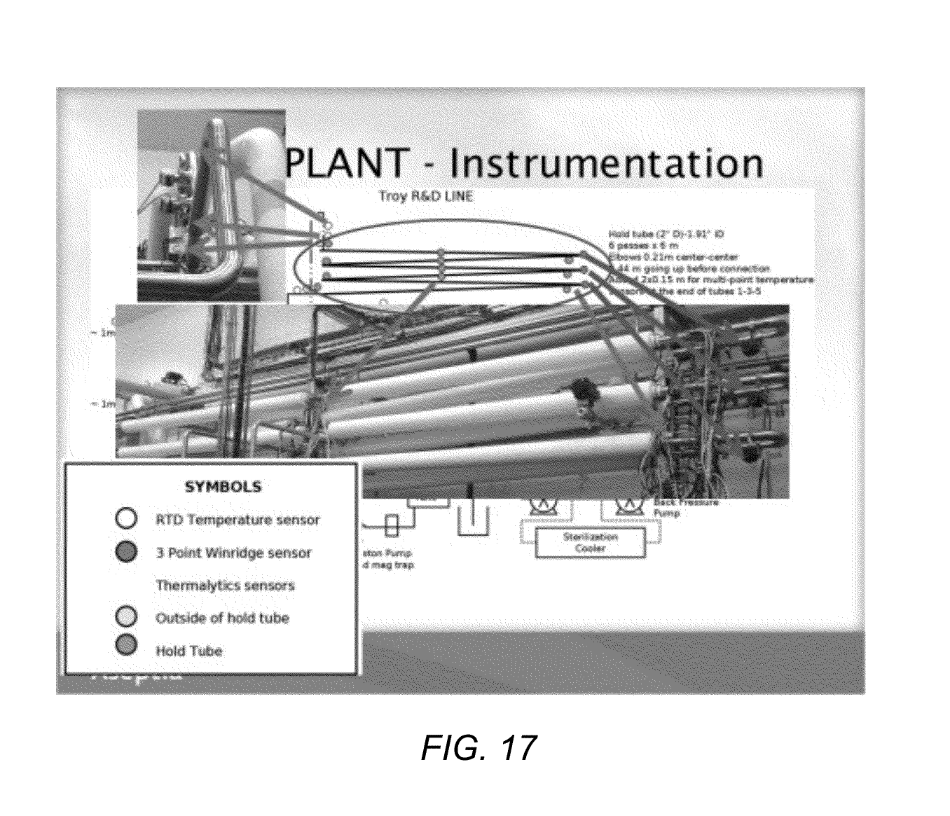

FIG. 17 illustrates an example plant, and plant instrumentation.

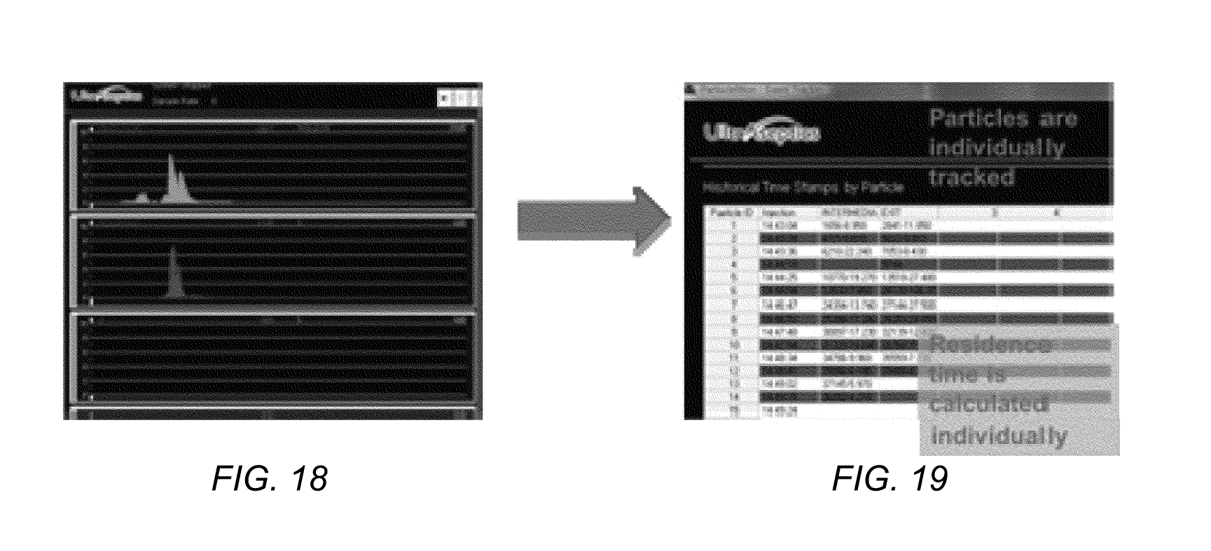

FIGS. 18-19 illustrate examples of time-temperature monitoring and reconstruction for each particle.

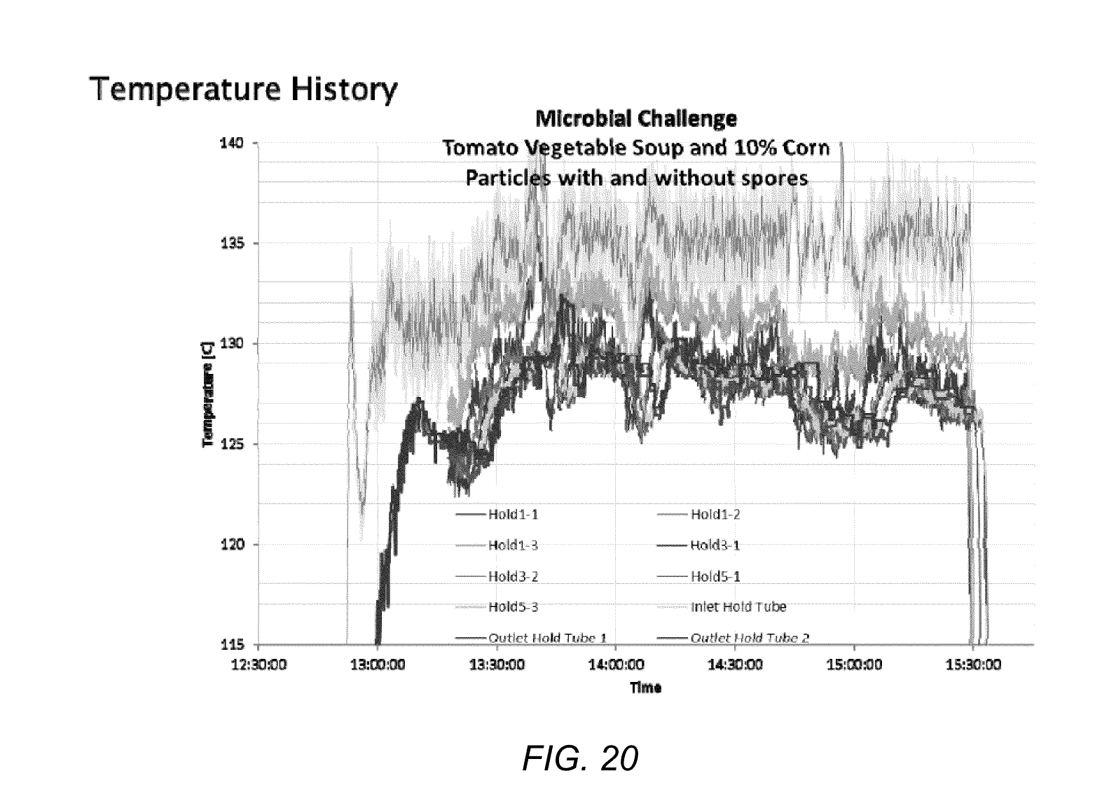

FIG. 20 illustrates an example temperature history.

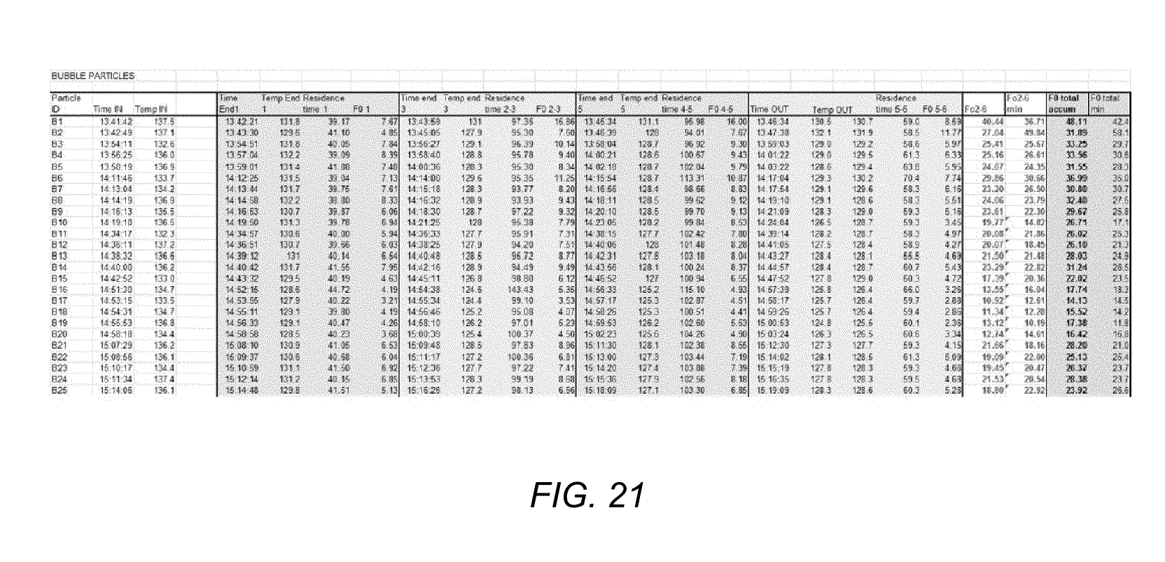

FIG. 21 illustrates reconstruction of temperature histories for each test particle that were carried out to observe each individual segment (F₀) accumulation for fluid, bulk and worst-case.

D) Secondary post-process confirmation implant/bio-validation of sterility by incubation of bacterial spores.

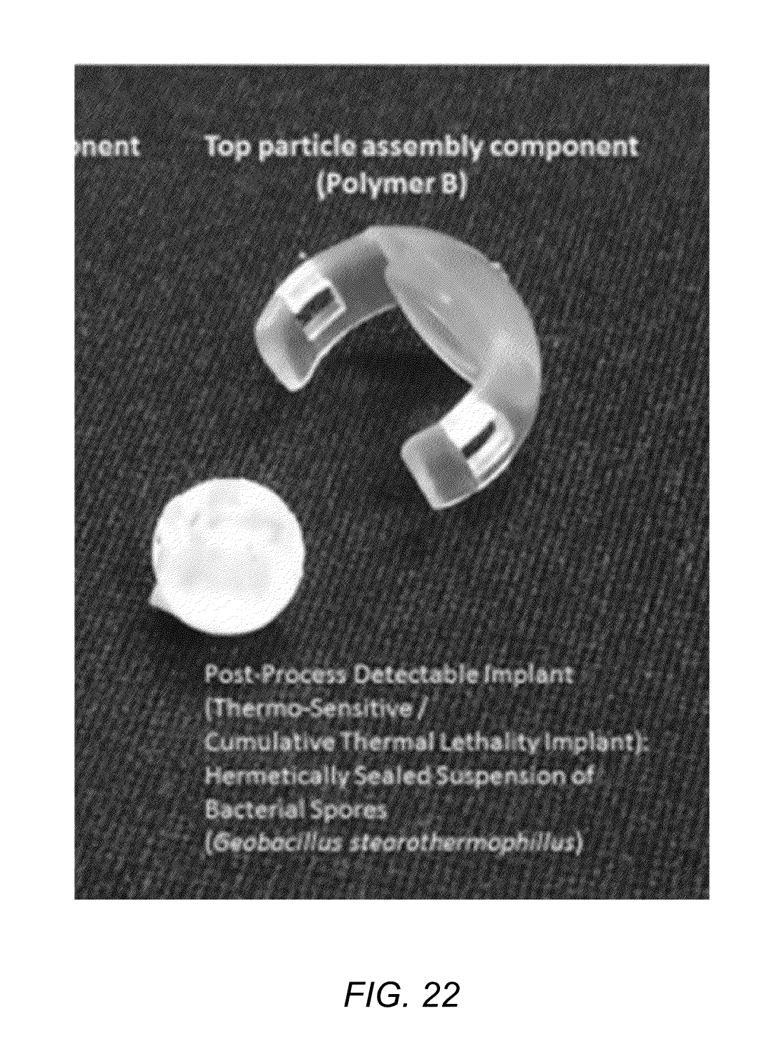

FIG. 22 illustrates an example of a top particle assembly component and a post-process detectable implant.

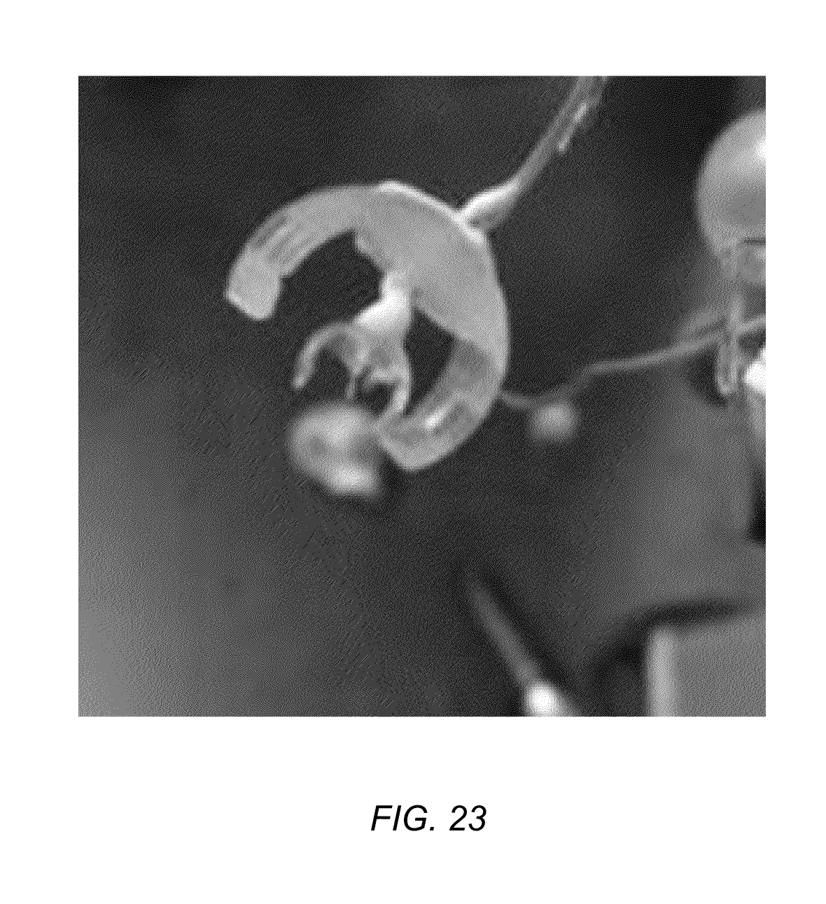

FIG. 23 illustrates another example.

E) Color changing indicator to determine inactivation or growth of bio-indicator organisms (such as bacterial spores, preferably Geobacillus stearothermophillus).

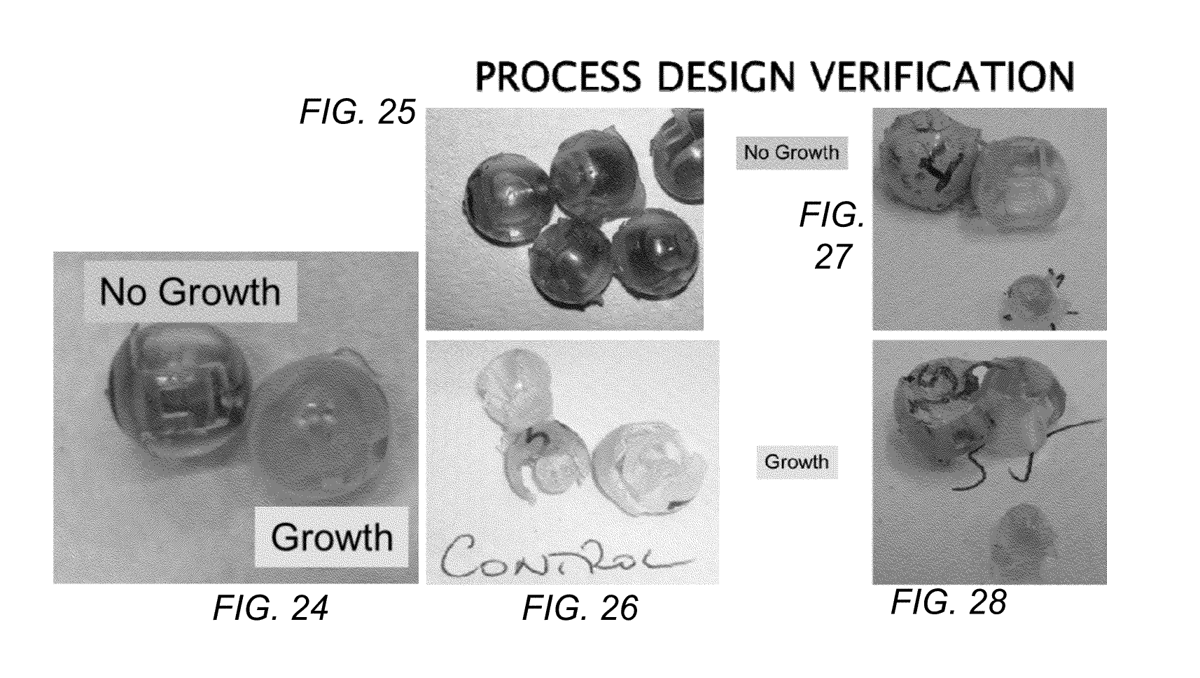

FIGS. 24-28 illustrate examples. In some embodiments a particle has a first color and a second color, wherein the particle changes from the first color to the second color in the presence of bio-indicator organisms. FIG. 24 shows a particle having a first color (“no growth”) and a particle having a second color (“growth”).

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

What is claimed is:

1. A fabricated device designed to simulate biomaterial products being processed using continuous flow thermal sterilization, comprising:
a polymer carrier structure with conservative flow and thermal;
an identifier for identifying individual particle shells using at least one of letters, numbers, symbols, and color codes;
at least one primary implant enabling tracking and residence time measurement while travelling through the processing system in real time;
at least one secondary implant containing at least one of viable microbial cells, viable microbial spores, enzymes, DNA, RNA, and other sub-cellular entities; and
at least one indicator enabling determination of viability or inactivation of at least one of the secondary implants following at least one of incubation and chemical treatment.

2. The fabricated device of claim 1, wherein the biomaterial products comprise food particles containing food.

3. The fabricated device of claim 1, wherein the polymer carrier structure with conservative flow is faster than any biomaterial particle contained in the biomaterial product.

4. The fabricated device of claim 3, wherein the biomaterial particles are food particles.

5. The fabricated device of claim 1, wherein the polymer carrier structure with conservative thermal is slower heating than any biomaterial particle contained in the biomaterial product.

6. The fabricated device of claim 1, wherein the identifier is at least one of a marking and a coding on the particle shell.

7. A method of determining proper processing of particle containing biomaterials, the method comprising:
preparing at least one simulated particle comprising the fabricated device of claim 1;
inserting the at least one simulated particle into a continuous flow thermal processing system capable of sterilization of particle containing biomaterials;
monitoring movement of the at least one simulated particle through the processing system using at least one monitoring detection station, sensor, or sensor array;
capturing the at least one simulated particle following insertion into the processing system and exposure to a representative thermal processing treatment;
incubating the at least one captured simulated particle for a sufficient time and at a sufficient temperature to cause growth or chemical state change of at least one biological entity; and
determining a proper processing status of the processed product by evaluating the growth or absence of growth or chemical change in a secondary implant.

8. The method of claim 7, wherein when the particle containing foods have been properly processed, the particle containing foods are sterile, and wherein the proper processing status is sterility of the processed product.

9. The method of claim 7, wherein the particle containing biomaterials comprise food comprising food particles.

10. A sterilized shelf stable biomaterial product obtained by implementing the principles and procedures defined by any one or more of claims 1 and 7.

11. The sterilized shelf stable biomaterial product of claim 10, wherein the biomaterial product is a food product.
